(19) United States
(12) Patent Application Publication
(10) Pub. No.: US 2015/0057648 A1
(43) Pub. Date: Feb. 26, 2015

(54) Laser Device and Method of Use

(71) Applicant: AngioDynamics, Inc., Latham, NY (US)

(72) Inventors: Kevin Swift, Brighton, MA (US); Benjamin Bell, Shrewsbury, MA (US); Brett Zubiate, Duxbury, MA (US)

(73) Assignee: AngioDynamics, Inc., Latham, NY (US)

(21) Appl. No.: 14/464,213

(22) Filed: Aug. 20, 2014

Related U.S. Application Data
(60) Provisional application No. 61/867,627, filed on Aug. 20, 2013.

(51) Int. Cl. A61B 18/22; A61B 18/24

(52) U.S. Cl. CPC A61B 18/22; A61B 18/24; A61B 2018/00577
USPC 606/15

(57) ABSTRACT

It is an object and advantage of this invention to provide an improved device and method that uses targeted laser wavelength to treat a diseased vessel. An advantage of this invention is targeted ablation of diseased vessels without harming non-target tissue. This new technique allows for a controlled ablation, may not require injection of tumescent anesthesia prior to treatment and may decrease unwanted or unintended side effects.

### CROSS REFERENCE TO RELATED APPLICATIONS

This application claims priority under 35 U.S.C. 119(e) to U.S. Provisional Application No. 61/867,627, filed Aug. 20, 2013, which is incorporated herein by reference.

### FIELD OF INVENTION

The present invention relates to a medical device and method for treating blood vessels, and more particularly to a laser treatment device and method for causing closure of varicose veins.

### BACKGROUND OF INVENTION

Veins are thin-walled and contain one-way valves that control blood flow. Normally, the valves open to allow blood to flow into the deeper veins and close to prevent back-flow into the superficial veins. When the valves are malfunctioning or only partially functioning, however, they no longer prevent the back-flow of blood into the superficial veins. As a result, venous pressure builds at the site of the faulty valves. Because the veins are thin walled and not able to withstand the increased pressure, they become what are known as varicose veins which are veins that are dilated, tortuous or engorged.

In particular, varicose veins of the lower extremities are one of the most common medical conditions of the adult population. Symptoms include discomfort, aching of the legs, itching, cosmetic deformities, and swelling. If let untreated, varicose veins may cause medical complications such as bleeding, phlebitis, ulcerations, thrombi and lipodermatosclerosis.

Traditional treatments for varicosities include both temporary and permanent techniques. Temporary treatments involve use of compression stockings and elevation of the diseased extremities. While providing temporary relief of symptoms, these techniques do not correct the underlying cause that is the faulty valves. Permanent treatments include surgical excision of the diseased segments, ambulatory phlebectomy, and occlusion of the vein through chemical or thermal ablation means.

Surgical excision requires general anesthesia and a long recovery period. Even with its high clinical success rate, surgical excision is rapidly becoming an outmoded technique due to the high costs of treatment and complication risks from surgery. Ambulatory phlebectomy involves avulsion of the varicose vein segment using multiple stab incisions through the skin. The procedure is done on an outpatient basis, but is still relatively expensive due to the length of time required to perform the procedure.

Chemical occlusion, also known as sclerotherapy, is an in-office procedure involving the injection of an irritant chemical into the vein. The chemical acts upon the inner lining of the vein walls causing them to occlude and block blood flow. Although a popular treatment option, complications can be severe including skin ulceration, anaphylactic reactions and permanent skin staining. Treatment is limited to veins of a particular size range. In addition, there is a relatively high recurrence rate due to vessel recanalization.

The use of embolic adhesives is also becoming more popular for treatment of varicose veins. Complications may include revascularization or incomplete vein closure that requires additional follow-up treatments and unwanted migration of the embolic adhesive.

Thermal ablation treatments, such as radiofrequency or laser energy, are becoming the most typical treatment for varicose veins. Endovascular laser therapy is a relatively new treatment technique for venous reflux diseases. Most prior art methods for laser ablation deliver the laser energy by a flexible optical fiber that is percutaneously inserted into the diseased vein prior to energy delivery. An introducer catheter or sheath is typically first inserted into the saphenous vein at a distal location and advanced to within a few centimeters of the saphenous-femoral junction of the great saphenous vein. Once the sheath is properly positioned, a flexible optical fiber is inserted into the lumen of the sheath and advanced until the fiber tip is near the sheath tip but still protected within the sheath lumen.

Known methods of thermal ablation using laser energy to treat varicose veins typically use with wavelengths between 810-1470 nm and targets absorption by the hemoglobin and/or water in the blood. As the hemoglobin and/or water in blood begin to rapidly heat as a result of energy absorption this creates a “thermal heat zone” or “heat bubble” inside the vessel. The “thermal heat zone” or “heat bubble” commonly leads to radiant or transient heating of the target zone, usually the inner cell lining of the varicose vein, and additionally non-target, healthy tissue surrounding the diseased vessel. One problem with radiant or transient heating is non-target tissue surrounding diseased vein wall, specifically the vein fascia containing nerves, may absorb the heat energy causing tissue temperature to rise above the pain and cell damage threshold of 45-50 degrees Celsius. This high absorption of energy by non-target tissue in turn causes unwanted symptoms in the patient, including vessel perforation, bruising, nerve damage, skin burns, patient pain, and general discomfort during and after treatment. To limit such symptoms tumescent injections are used prior to treatment.

Tumescent injections, typically a fluid mixture of lidocaine and saline with or without epinephrine, are administered along the entire length of the great saphenous vein using ultrasonic guidance and the markings previously mapped out on the skin surface. The typical tumescent injection process is time consuming and may take up to 30 minutes to complete. The tumescent injections perform several functions, including pain relief; acting as a thermal barrier between the vein wall and surrounding tissue, and a compressive force to reduce the vein diameter providing better contact with the ablation device. The anesthesia inhibits pain caused from application of laser energy at higher wavelengths to the vein resulting in tissue temperatures to rise above the pain and cell damage threshold of 45-50 degrees Celsius. The tumescent injection also provides a barrier between the vessel and the adjacent tissue and nerve structures, which restricts some of the heat damage to within the vessel. However, this barrier does not prevent all non-target tissue damage. As described in more detail below, an object of the current invention is to eliminate the need for tumescent injections. Further, patients can still experience pain and discomfort from undergoing endovenous laser treatment, especially if the tumescent administered is insufficient. Lastly, the requirement of tumescent anesthesia adds to the economic cost of the overall procedure.

With some of the prior art treatment methods, contact between the energy-emitting face of the treatment device and the inner wall of the varicose vein is recommended to ensure complete collapse of the diseased vessel. For example, U.S. Pat. No. 6,398,777, issued to Navarro at al, teaches either the means of applying pressure over the laser tip or emptying the vessel of blood to ensure that there is contact between the vessel wall and the fiber tip. One problem with direct contact between the laser fiber tip and the inner wall of the vessel is that it can result in vessel perforation and extravasation of blood into the perivascular tissue. This problem is documented in numerous scientific articles including “Endovenous Treatment of the Greater Saphenous Vein with a 940-nm Diode Laser: Thrombotic Occlusion After Endoluminal Thermal Damage By Laser-Generated Steam Bubble” by T. M. Proebstle, MD, in Journal of Vascular Surgery, Vol. 35, pp. 729-736 (April, 2002), and “Thermal Damage of the Inner Vein Wall During Endovenous Laser Treatment: Key Role of Energy Absorption by Intravascular Blood” by T. M. Proebstle, MD, in Dermatol Surg, Vol 28, pp. 596-600 (2002), both of which are incorporated herein by reference. When the fiber contacts the vessel wall during treatment, intense direct laser energy is delivered to the vessel wall. Conversely, by preventing direct contact between fiber and vein wall the energy is delivered to the vessel wall by indirect or radiant thermal energy from the gas bubbles caused by heating of the blood. Laser energy in direct contact with the vessel wall causes the vein to perforate at the contact point and surrounding area. Blood escapes through these perforations into the perivascular tissue, resulting in post-treatment bruising and associated discomfort.

Another problem created by the prior art methods involving contact between the fiber tip and vessel wall is that inadequate energy is delivered to the non-contact segments of the diseased vein. Inadequately heated vein tissue may not occlude, necrose or collapse, resulting in incomplete treatment.

Additionally, most conventional endovenous laser treatments use forward firing lasers which require high power densities to boil or heat the blood, creating bubbles which are necessary for 360 degree circumferential treatment of the targeted vein. High power densities can cause perforations, bruising, nerve damage, thermal damage to non-targeted tissue and other complications causing the patient additional pain. High power densities also cause charring of blood on the fiber tip.

Therefore, it would be desirable to provide an endovascular treatment device and method that applies lower power density energy directly to the tissue lining the vessel wall which can be uniformly applied to the vessel while avoiding thermal damage to non-targeted tissue.

It is also desirable to provide an endovascular treatment device and method which protects the optical fiber fom direct contact with the inner wall of vessel during the emission of laser energy to ensure consistent thermal heating across the entire vessel circumference thus avoiding vessel perforation and/or incomplete vessel collapse.

It is another purpose to provide and endovascular treatment which eliminates the need for tumescent anesthesia thus avoiding the time, pain and cost associated with the administration of tumescent.

It is another purpose to provide an endovascular treatment device and method which decreases peak temperatures at the working end of the fiber during the emission of laser energy thus avoiding the possibility of fiber damage and/or breakage due to heat stress caused by thermal runaway.

It is yet another purpose to provide an endovascular treatment device and method which is fast, effective and low in cost enabling the use of existing laser generator capital equipment.

Various other purposes and embodiments of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description of the Invention.

### SUMMARY OF INVENTION

According to one aspect of the present invention, an endovascular laser treatment device for causing closure of a blood vessel is provided. The treatment device uses an optical fiber having a core through which a laser light travels and is adapted to be inserted into a blood vessel. A cladding layer is arranged around the core such that the laser energy is maintained within the core. The fiber core may be etched, scored, cut, or otherwise abrasively altered such that slits or grooves are placed into the fiber core. At a distal end portion of the device, the cladding layer may have slits, holes, or openings to expose the core. The power density of the laser energy escaping through the etching of the core and slits of the cladding may be controlled by the variable pitch or surface area of the etches and slits along the ablation zone. It is an object of this invention to provide an energy device capable of 360 degree, side, radial, or circumferential thermal ablation of the blood vessel. The distal end portion of the device may be coaxially surrounded by a sleeve, diffusor, or spacer which aids in the emission of the laser energy as it passes through the slits.

As described in more detail below, the energy delivery device may provide substantially lower power density emission, as compared to traditional forward firing energy deliver devices currently known in the art. The reduced power density emission is accomplished by increasing the surface area of exposed fiber core through which laser energy may be emitted. The exposed surface area, or ablation zone, is created by removing the cladding and optionally a portion of the core, in a pattern of etches or slits near the distal portion of the fiber. The pattern may include etches which are angled relative to the longitudinal axis of the device and which vary in pitch, width and/or spacing. The reduced power density lowers peak temperatures in the blood vessel and advantageously prevents thermal runaway, unwanted radiate heating to healthy tissue, and device damage. The reduction in power density also reduces the possibility of vessel perforations, prevents bruising, post-operative pain and other clinical complications.

In another embodiment of the invention, the distal end portion is further coaxially surrounded by a spacer. The spacer may take the form of an expandable member, such as a balloon or arms, a non-expandable member, such as a diffuser cap, or another spacer type element that is intended to keep the ablation zone of the fiber from direct contact with the vein wall. If the spacer is an expandable balloon this may prevent the fiber from coming into direct contact with the blood vessel and aids in the emission of laser energy to evenly treat the vessel wall. The balloon spacer and fiber embodiment includes a dual lumen outer shaft having an inflation/deflation lumen and a lumen sized for passage of the fiber.

A method for causing closure of a blood vessel is provided. The method involves inserting into a blood vessel an optical fiber having etches in the fiber core and slits or removed cladding layer at a distal portion of the device. Advantageously, the etching and slits enable a controlled power density emission along the ablation zone at the distal end of the fiber. The power density can be controlled so that the modality of treatment is not radiant heating, as currently used in the art by both laser and RF devices, but rather direct and controlled heating of the inner layer of endothelial cells lining the vein wall. The controlled heating of the inner layer of endothelial cells lining the vein wall reduces the possibility of vessel wall perforations and bruising. Therefore, this method may not require the administration of tumescent anesthesia before the procedure.

A method for causing closure of a blood vessel using a balloon spacer is also provided. In this embodiment, the distal end portion is also surrounded by a balloon, which, when in an inflated state, is in contact with the vessel wall. An outer shaft is inserted into the blood vessel, the outer shaft providing an inflation/deflation lumen and a lumen for passage of the fiber. The inflation/deflation lumen passes a gas or liquid, including but not limited to carbon dioxide gas, to inflate the balloon once the balloon is within the treatment site. When laser energy passes through the slits, the balloon further aids in radial treatment of the blood vessel while preventing the fiber from coming in direct contact with the vessel wall. The administration of tumescent anesthesia is not required in this method.

### BRIEF DESCRIPTION OF DRAWINGS

FIG. 1 represents a perspective view of the one embodiment of the side-firing fiber optic laser device and laser generator.

FIG. 2A is a longitudinal plan view of the distal section of the optical fiber assembly.

FIG. 2B is a cross-sectional view of FIG. 2A taken along line A-A.

FIG. 2C is a cross-sectional view of FIG. 2A taken along line B-B.

FIG. 3 is a partial side view of the distal end of the optical fiber and sleeve prior to manufacture.

FIG. 4A is a longitudinal plan view of the distal section of the optical fiber assembly.

FIG. 4B is a side view of one embodiment of the distal section of the optical fiber.

FIG. 4C is a side view of another embodiment of the distal section of the optical fiber.

FIG. 5A is a side view of yet another embodiment of the distal section of the optical fiber.

FIG. 5B is a cross-sectional view of FIG. 5A taken along line A-A.

FIG. 5C is a side view of another embodiment of the distal section of the optical fiber showing helical grooves.

FIG. 5D is a side view of another embodiment of the distal section of the optical fiber showing slit grooves.

FIG. 5E is a side view of another embodiment of the distal section of the optical fiber showing circular shaped grooves.

FIG. 5F is a side view of another embodiment of the distal section of the optical fiber showing longitudinal grooves.

FIG. 5G is a side view of another embodiment of the distal section of the optical fiber showing helical shaped grooves with a variable pitch.

FIG. 5H is a side view of another embodiment of the distal section of the optical fiber showing annular shaped grooves with a variable groove spacing.

FIG. 5I is a side view of another embodiment of the distal end of the optical fiber showing helical shaped grooves with a variable pitch and a sensor.

FIG. 5J is an image showing the laser energy being emitted from the distal section of the optical fiber.

FIG. 5K is an image showing coagulated blood accumulated on the distal section of the optical fiber after it has been used to ablate tissue.

FIG. 5L is an image of prior art forward-firing laser showing coagulated blood accumulated on the distal section of the optical fiber after it has been used to ablate tissue.

FIG. 6 is a schematic of another embodiment of the device having an expandable member located near the distal section.

FIG. 7 is a partial side view of the embodiment of FIG. 6 in a non-deployed state.

FIG. 8A is a partial side view of the embodiment of FIG. 6 in a deployed state.

FIG. 8B is a cross-sectional view of FIG. 8A taken along line A-A.

FIG. 8C is a cross-sectional view of FIG. 8A taken along line B-B.

FIG. 8D is a cross-sectional view of FIG. 8A taken along line C-C.

FIG. 8E is an image of the embodiment described in FIG. 6 showing laser energy being emitted.

FIG. 9 is a flowchart depicting method steps for performing endovenous laser treatment using one embodiment of the device.

FIG. 10 is a flowchart depicting method steps for performing endovenous laser treatment using another embodiment of the device.

### DETAILED DESCRIPTION OF THE INVENTION

The following descriptions and the associated drawings describe exemplary embodiments in the context of certain exemplary combinations of elements and/or functions; it should be appreciated that different combinations of elements and/or functions can be provided by alternative embodiments without departing from the scope of the appended claims. In this regard, for example, different combinations of elements and/or functions than those explicitly described above are also contemplated.

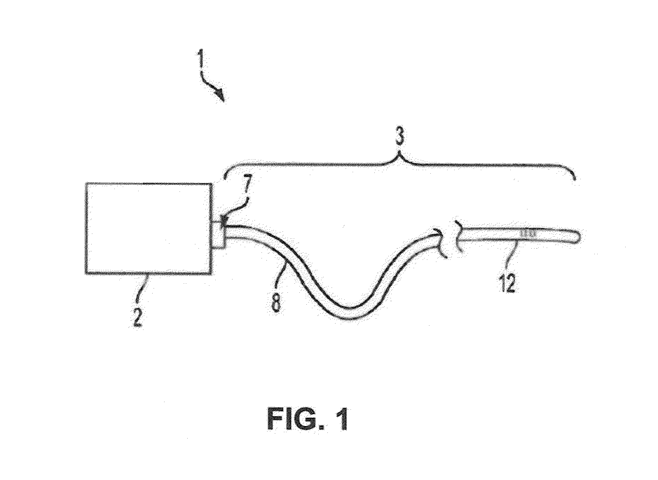

A first embodiment of the present invention is shown in FIGS. 1-4C. The endovascular treatment device 1 shown in FIG. 1 comprises a generator 2, an optical fiber 3 having a distal portion 12 and a proximal portion 8, and a proximal connection 7 from the optical fiber to the laser generator. The device may operate in a range of different energy wavelengths, including but not limited to, 200 nm-2500 nm, depending on the laser generator. The proximal connection 7 may have a SMA or similar-type connector, which can be attached to the end of the proximal portion 8 of the fiber 3.

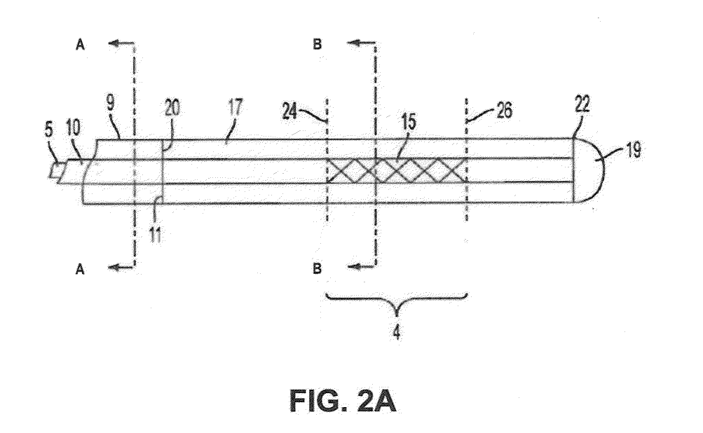

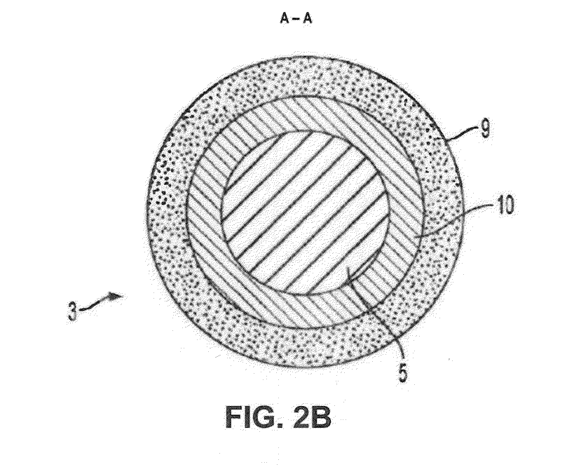

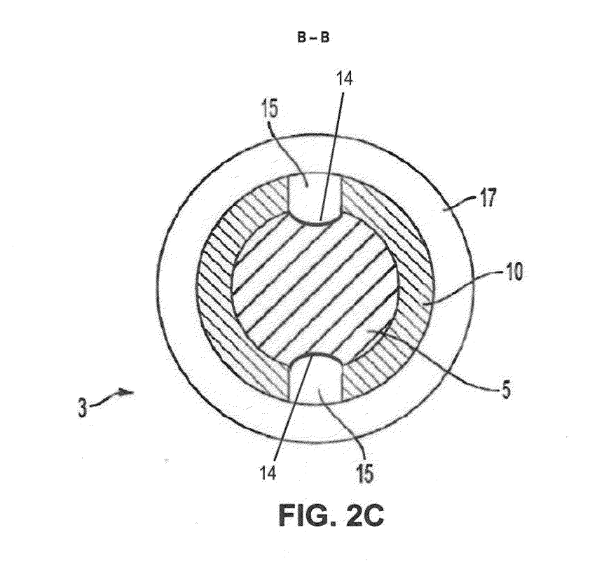

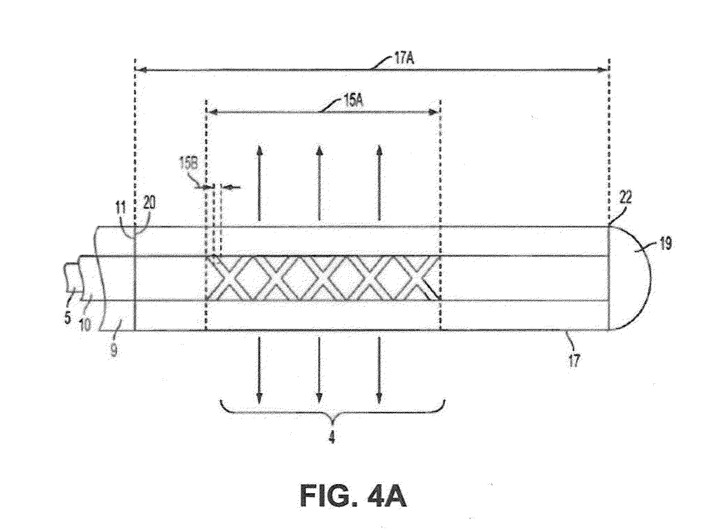

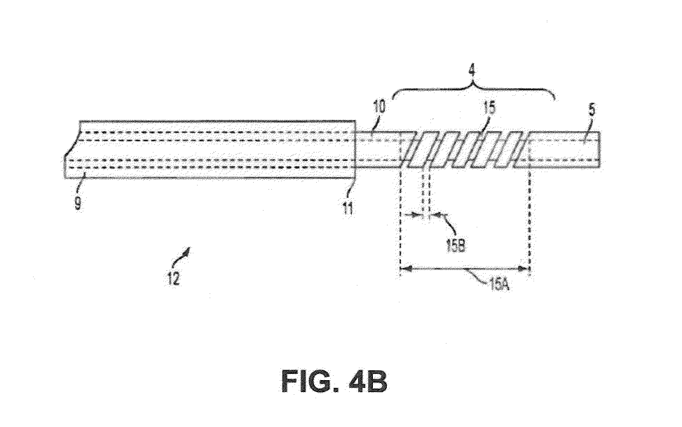

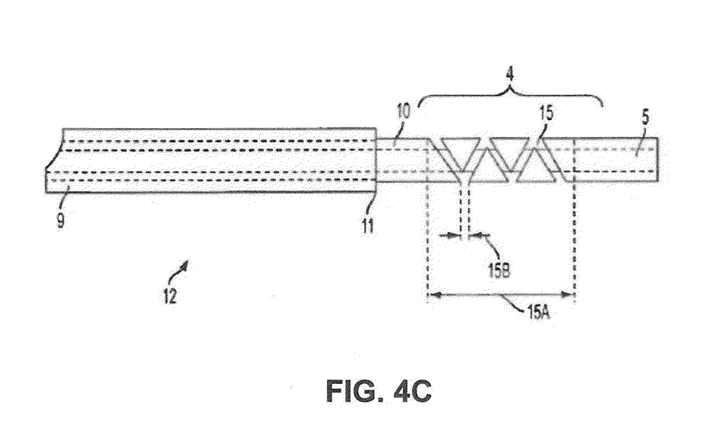

FIG. 2A shows a longitudinal plan view of distal section 12 of the optical fiber of the first embodiment. This embodiment is comprised of a fiber core 5 coaxially surrounded by a cladding layer 10, and a protective jacket 9 coaxially surrounding the cladding layer 10. The radial energy emitting section 4 us comprised of the core 5 with etches, a cladding layer 10, slits 15 of removed cladding, and an outer sleeve 17. The sleeve 17 may be a fused quartz ferrule or diffuser sleeve used to disperse the laser energy as it passes out of the slits 15. The sleeve 17 may have a desired length of 5-20 mm from a proximal edge 20 of the sleeve 17 to a distal edge 22 of the sleeve 17. The proximal edge 20 of the sleeve 17 may abut the distal edge 11 of the jacket 9. This is where the jacket 9 ends and the sleeve 17 begins. The sleeve 17 has a longitudinal through lumen 18 with an inner diameter so that it can coaxially surround the exposed distal end section 12, as shown in FIG. 4B. The distal end 22 of the sleeve 17 may abut the distal tip 19. The distal tip 19 may be a cap or plug made from similar material as the sleeve 17 and may be attached to the sleeve 17 by various methods, including but not limited to, adhesive, welding, or fusing welded or otherwise attached to the sleeve 17 by known methods in the art. Alternatively, the distal tip 19 may be formed by fusing the distal end of the fiber core 5 with the sleeve 17, such a technique is described in detail in U.S. application Ser. No. 12/100,309, entitled “DEVICE AND METHOD FOR ENDOVASCULAR TREATMENT FOR CAUSING CLOSURE OF A BLOOD VESSEL”, which is incorporated herein by reference. The distal tip 19 may be a convex shape as shown, or may form various other configurations, such as concave, flat, pointed, or other tip configurations known in the art. A convex distal tip 19 may be advantageous because it may help prevent unwanted vessel perforations or punctures during insertion of the fiber into a tortuous varicose vein.

As known in the art, cladding 10 is intended to prevent light waves from escaping or being emitted from the core 5. Light energy travels in the path of least resistance. As light waves travel down the core 5 and encounter the etching of the core 5 and slits 15 of the cladding 10 the waves will begin to escape through the grooves and lists and be emitted into the surrounding vessel. The majority of the light energy will be delivered from the radial energy emitting section 4 because this section of the fiber has the most proximal exposed core surface area which permits light energy to pass through. However, depending on the power of the laser energy and the path of the light waves it is possible that a small percentage of light energy may also be emitted from the distal tip 19, as shown and described in more detail below. The light escaping from the distal tip 19 is not intended to have the power density sufficient to ablate tissue. Rather it is merely the remaining light energy—which will typically be around less than 5% of the overall light energy—that has not escaped along the radial energy emitting section 4.

FIG. 2B shows a cross-sectional view along line A-A′ of FIG. 2A which represents the configuration of the fiber 3 including the core 5, cladding 10, and jacket 9. As disclosed herein, the fiber core 5 may range from 200-1000 microns in diameter. Preferably, the core 5 may be 400 or 600 microns. The cladding layer 10 creates a barrier which the laser energy cannot penetrate, thus causing the energy to move longitudinally through the fiber 3 to a radial energy emitting section 4 of the fiber 3. The jacket 9 prevents the fiber from breaking during use or during transport. The jacket 9 may also have markings on it as described in more detail below.

FIG. 2C represents a cross-sectional view of radial energy emitting section 4 line B-B′ of FIG. 2A. The radial energy emitting section 4 comprises the core 5 and grooves 14 in core 5, cladding layer 10 and slits 15 in cladding layer 10, and sleeve 17. The cladding layer 10 may have slits 15 or openings. The slits 15 align with the grooves 14 in the core 5. Grooves 14 are etched into the core 5 using a laser or other known technique in the art. The depth of the grooves 14 may vary depending on the desired resulting power density. It is an intention of this embodiment that the grooves 14 extend toward the central axis of the fiber core 5. The grooves 14 will generally have a semispherical geometry. The grooves 14 will create a surface in the core 5 so when light energy hits the grooves 14 the angle of refraction created by the grooves 14 permit the light energy to escape. The index of refraction (n) for fused silica glass ranges from 1.4-1.5 in the wavelengths of 800 nm to 2000 nm, respectively. Therefore, the grooves 14 are sized such that the light energy, in the wavelength ranges 800 nm-2000 nm, when refracted creates angles ranging from 40-45 degrees; enabling light energy to escape through the sleeve 17.

In one exemplary aspect, the fiber may be a 600 micron fiber, the core 5 may be about 0.600 mm+/−0.010 mm in diameter and the thin cladding layer 10 may have 0.030 mm+0.005/−0.010 mm outer diameter. In another aspect, the fiber may be a 400 micron fiber, the core 5 having a 0.400 mm+/−0.010 mm diameter and a cladding of 0.030 mm+0.005/−0.010 mm. The fiber 3 may be comprised of a silica based core 5 and a polymer cladding layer 10 (e.g., fluoropolymer). In another aspect, the optical fiber 3 may be comprised of a glass core 5 and a glass (e.g., doped silica) cladding layer 10. For this embodiment, the outer surface of the cladding layer 10 and inner surface of the sleeve 17 may have an interference fit.

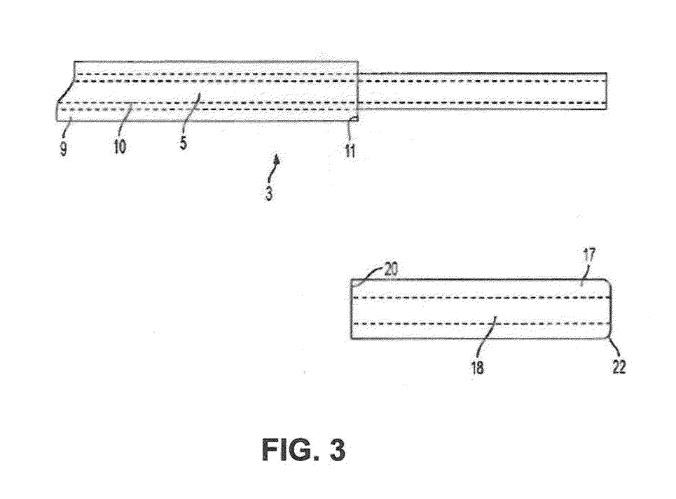

Referring to FIG. 3, which shows the components before assembly of the radial energy emitting section 4 of the device 1, the fiber 3 is shown with the protective jacket 9 partially removed from the distal end 11 the fiber 3. The sleeve 17, in this embodiment is made from glass or silica, has an inner lumen 18 which extends from a proximal end 22 to a distal end 20. Prior to assembly of the fiber and sleeve 17, the cladding layer 10 is partially removed to form the slits 15 by known methods in the art, and as described below and seen in FIG. 4A-FIG. 4C. Once the slits 15 have been formed in the cladding layer 10 the fiber core 5 may then be etched with the desired grooves, as described above. First, removing the cladding 10 to create slits 15 prior to etching the core 5 ensures that the cladding material 10 does not mix or contaminate the core 5 during the etching process. Next, the sleeve 17 is secured to the fiber 3 so that it coaxially surrounds the portion of the fiber 3 having the slits 15. The proximal end 20 of the sleeve 17 abuts adjacent to the proximal end 11 of the jacket 9. As discussed above, the distal end 22 of the sleeve 17 is joined together or created into the distal tip 19.

Referring to FIG. 4A-FIG. 4C, which depicts the distal section of the fiber, the dimensions and geometry of the cladding 10 slits 15 and etching 14 in core 5 may be in any configuration to allow radial emission of laser energy without departing from the scope of the invention. For this embodiment, the dimensions and surface area of the grooves 14 and slits 15 will directly impact the resulting power density along the radial energy emitting section 4. For example, the resulting power density along the radial energy emitting section 4 can be controlled by altering and customizing the size, placements and number of slits 15. By way of example, the total slit length 15A, slit width 15B, the pitch of the slits relative to the longitudinal axis of the fiber may be varied to form unique slit patterns designed to deliver optimal energy densities along the treatment zone. Adjusting the overall dimension and geometry of the slits 15 will directly impact the amount of light energy leakage or radial light energy dissipation, power density delivered along treatment section, direction of light energy, and power density that will escape from distal end 19 of the fiber 3. The double helical configuration of the slit length 15A, as seen in FIG. 4A, may ensure a radial or complete and even 360 degree treatment of the vessel. A double helix slit 15 configuration consists of two congruent helices with the same axis that differ by translation along the axis.

FIGS. 4B and 4C show the distal end section 12 of the fiber with alternative slit 15 configurations from the previous embodiment. The jacket 9 has been removed and slit 15 pattern is created before the sleeve 17 is attached. The radial energy emitting section 4 of FIG. 4B shows a spiral or cork-screw slit configuration, while the radial energy emitting section 4 of FIG. 4C depicts a zigzag or triangle pattern of slits 15. The slits 15 may be formed using various techniques. For example, one method of creating the slits 15 is to remove only sections of the cladding 10 along the distal end, as seen in FIG. 4B-4C. The slits 15 may take may different forms and patterns, including but not limited to, helical, spiral, radial, circular, zigzag, wedge-shaped or dotted.

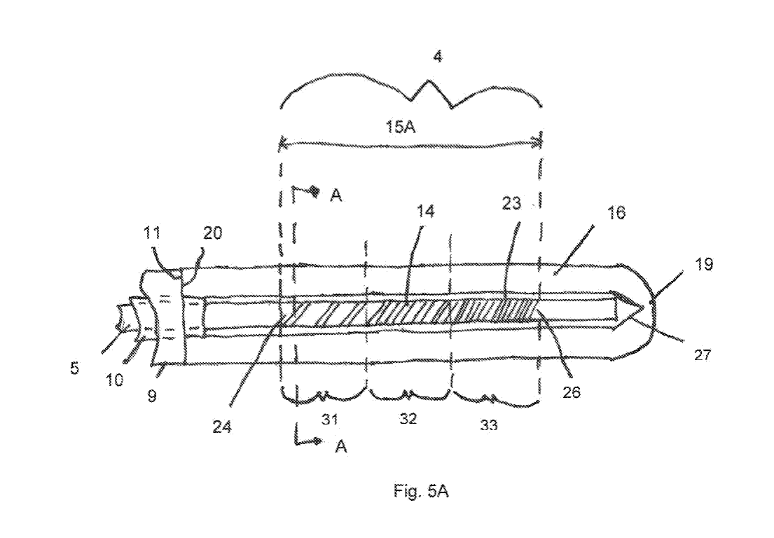

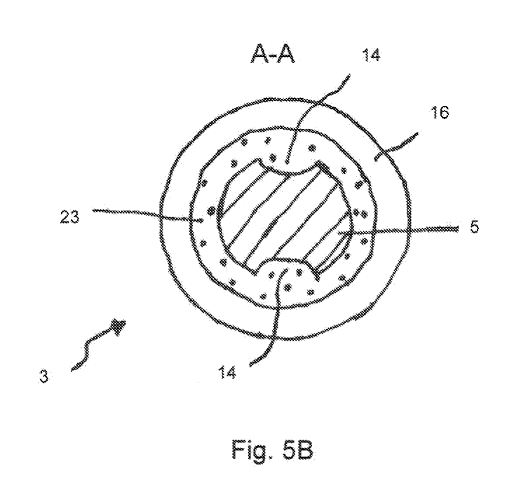

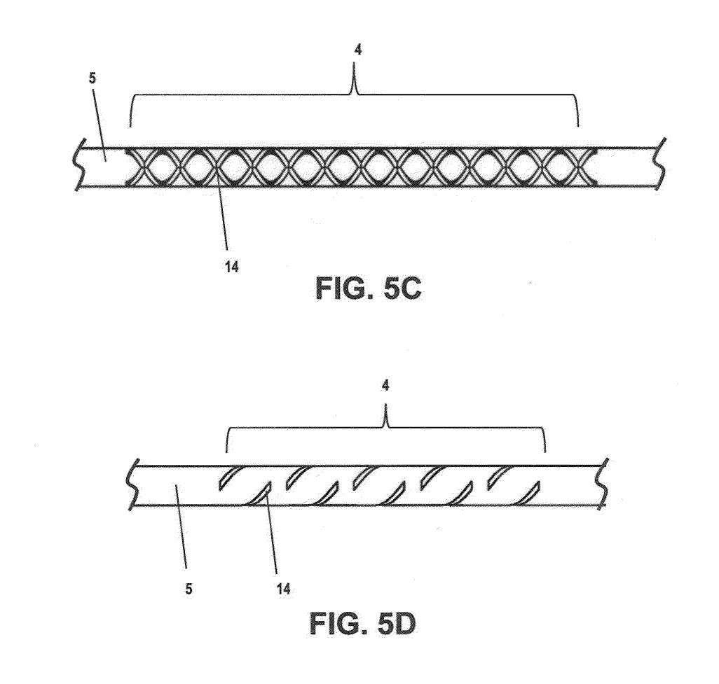

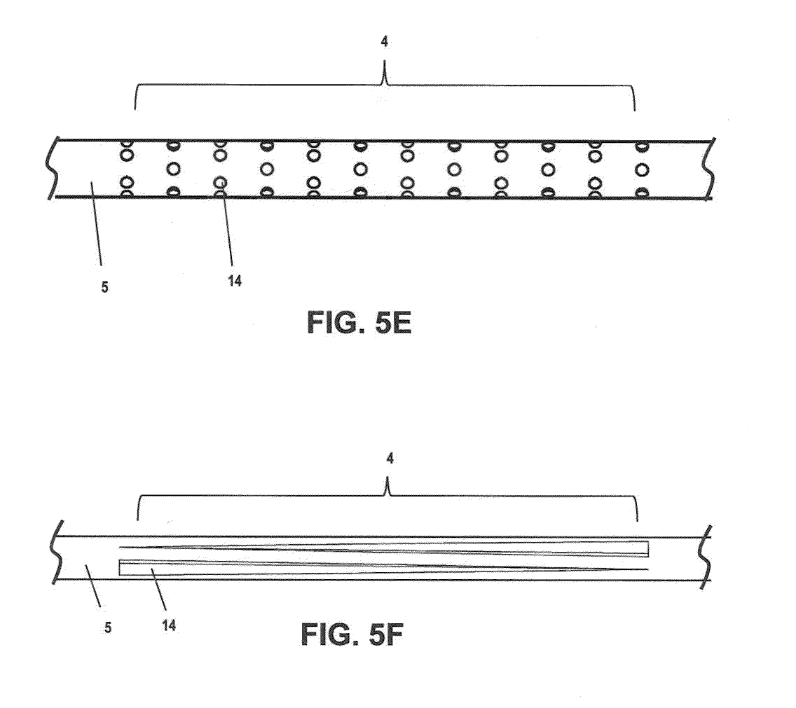

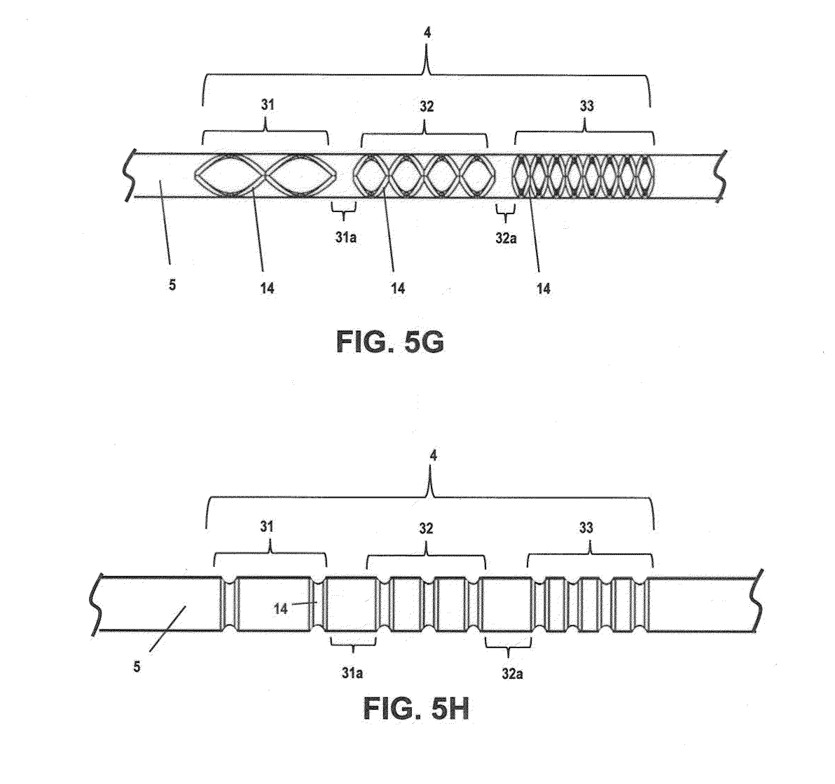

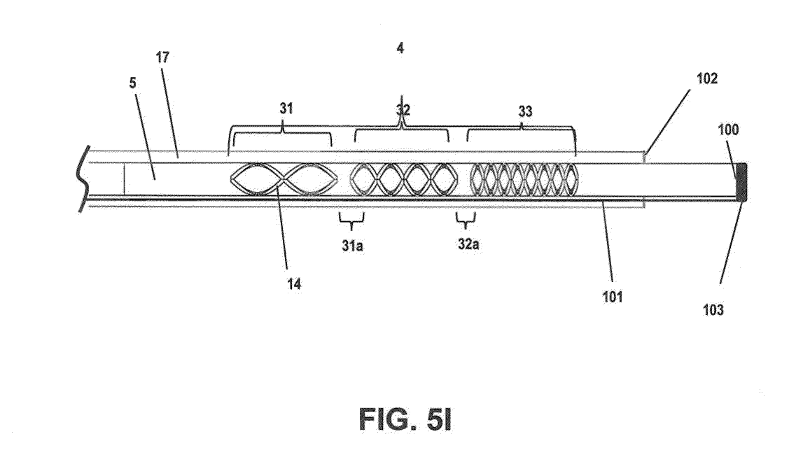

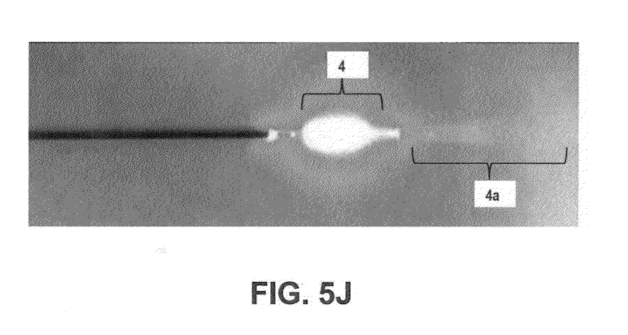

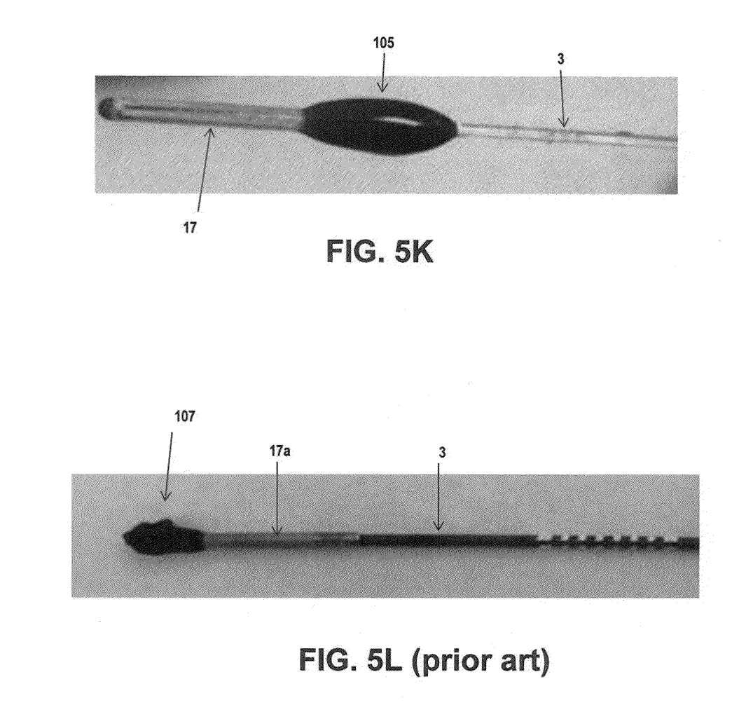

Referring now to FIG. 5A-5B, another embodiment of the device is shown. A related problem with endovascular laser treatment of varicose veins using a conventional fiber device is fiber tip damage during laser energy emission caused by localized heat build up at the working end of the fiber, which may lead to thermal runaway. Thermal runaway occurs when temperature at the fiber tip reaches a threshold where the core and/or cladding begin to absorb the laser radiation. As the fiber begins to absorb the laser energy it heats more rapidly, quickly spiraling to the point at which the emitting face begins to burn back like a fuse. One cause of the heat build up is the high power density at the emitting face of the fiber. A conventional fiber includes a cladding layer immediately surrounding the fiber core. Laser energy emitted from the distal end of the device may create thermal spikes with temperatures sufficiently high to cause the cladding layer to burn back. By removing the cladding 10 for a selected distance from the distal end of the core 5, the possibility of burn back of the cladding 10 is eliminated. In this embodiment the cladding layer 10 may be completed removed from distal section of core 5. Grooves 14 are then etched directly into the core 5 at variable pitches. The grooves 14 may be etched into the core 5 using a laser or other known technique in the art. The depth and pitch of the grooves 14 may vary depending on the desired power density. It is an intention of this embodiment that the grooves 14 extend toward the central axis of the fiber core 5. As shown in FIG. 5B, which represents a cross-sectional view along line 10a in FIG. 5A, the grooves 14 may generally have a semispherical geometry.

After the grooves 14 have been etched into the core 5, an outer cap 16, which may be made from glass or fused silica similar to the sleeve described above, is placed over the core 5 and attached to the jacket 9 using an adhesive or other known method in the art. The outer cap 16 gives the fiber 3 a convex distal tip 19. This convex shaped tip 19 helps ease the advancement of the fiber. The outer cap 16 is sized such that there is a space between the outer cap 16 and core 5 creating an air gap 23 around the distal end of core 5. The light energy remains inside the fiber core 5 as a result of the cladding layer 10 and the air gap 23 which acts as an additional cladding layer only for the section of core 5 that does not have any grooves 14. The light energy remains inside the fiber core 10 as a result of the cladding layer 10 and the air gap 23 which acts as an additional cladding layer.

The air gap 23 will be present and fill this void as seen in FIG. 5B, representing a cross-sectional view of FIG. 5A taken along line A-A. In this embodiment, air will have a lower refractive index than the outer cap 16. For example, the outer cap 16 may be comprised of a fused silica or other glass material that has a similar index of refraction as the core 5. The air gap 23 functions as an additional or secondary cladding layer. The grooves 14 cause a void along the smooth surface of the core 5. The voids provide a necessary interface that will expel the light waves from the core 5 through the air 23 and subsequent cap 16. The voids introduce sharp angled surfaces into the core 5 that will be able to surpass the critical angle established by the indices of refraction of the interface between the air 23 and core 5 that would have otherwise been unachievable. Some light waves will hit the grooves 14 at an angle less than the critical angle for total internal reflection to occur. The critical angle of incidence is a function of the indices of refraction for the two materials at the interface; in this case, the two materials are core 5 and air 23. Once outside the core 5, the light waves are able to be transmitted through the outer cap 16 because its index of refraction is higher than that of the air gap 23 which prevents total internal reflection.

The outer cap 16 may also has concave 27 shape along its inner wall at its distal end. The inner wall concave shape 27 may facilitate reflection of any remaining forward emitting light back through the core 5. As the laser energy travels down the fiber 3 toward the distal tip 19, the small percentage of forward firing light energy will reach the concave shape 27 and reflect the light back towards the core 5 and thereby reduce the amount of light passing through the distal tip 19 of the outer cap 16.

It is an advantage of this invention that the power density of the laser energy emitted along the radial energy emitting section 4 can be precisely controlled using variable pitches of the grooves 14. It is intended that this device will have a lower overall power density that what is currently used in forward firing lasers in the art but still have enough power density to cause thermal death to the inner cell wall of the target vein. The purpose of lowering the overall power density is to prevent unwanted vessel wall perforations or unwanted radiant heating that damages healthy tissue surrounding the target vessel. Currently tumescent anesthesia is used in part to act as a heat barrier between the energy device and the healthy surrounding tissue to decrease this unwanted radiant heating of non-targeted tissue. This device may solve the problem of unwanted radiant heating and not require the use of tumescent by controlling the amount of power density and light escaping the fiber along the radial energy emitting section 4.

By controlling the groove 14 pitch, groove size, groove 14 depth, groove 14 surface area and number of the grooves 14 along the radial energy emitting section 4 it will be possible to control and/or customize the power density of the emitted light energy along the entire length of the radial energy emitting section 4. Light energy travels in the path of least resistance so the amount of energy that is released along the radial energy emitting section 4 through the proximal edge 24 of the radial energy emitting section 4 is generally greater than the energy being released at the distal edge of the slits 26, for any given uniform slit pattern. In other words, there will be less available light energy to escape through the grooves 14 closer to the distal edge 26 of the radial energy emitting section 4. By varying the spacing, pitch, and other slit pattern characteristics, the energy emitted along the length of the emitting section 4 can be controlled. The proximal edge 24 of the radial energy emitting section 4 has grooves 14 that are spaced apart and few in number. As the groove 14 pitch moves towards the distal edge 26 the grooves 14 and pitch will become more numerous and closer together with a steeper pitch. The reason for increasing number of grooves 14 towards the distal edge 26 is to allow the maximum available light energy to escape in an effort to equalize the amount of light energy escaping along the radial energy emitting section 4. It is an intention of this device that the power density along the length of the radial energy emitting section 4 will be equal and sufficient enough to generate heat in the range of the 45-50 C at the vessel wall, the cell death threshold, but insufficient to cause unwanted radiant heating of non-target tissue, and thereby eliminating or minimizing the need for tumescent anesthesia.

The grooves 14 may be configured in any configuration stated above, but in this embodiment they are helical and have a groove pattern length 15A of approximately up to 15 mm. Furthermore, the groove pattern length 15A is comprised of a first or proximal zone 31, a second or intermediate zone 32, and a third or distal zone 33. The three zones preferably divide the groove length 15A into three equal sections. The zones are created to release a uniform radial band of laser energy. Therefore the grooves 14 will be configured so that the energy output of the first zone will equal the energy output of the second zone which will equal the energy output of the third zone 33. As seen in FIG. 5A, the number of grooves 14 may increase from the first zone 21 to the third zone 33, thereby controlling the power density of the laser energy being emitted. The first zone 31 may have the least number of grooves 14 to prevent the majority of the laser energy from escaping and to facilitate more laser energy traveling further down the fiber 3. The second zone 32 may have a greater number of grooves 14 than there are in the first zone 31, but a lesser number of grooves 14 than there are in the third zone 33. The third zone 33, which is close to the distal most tip 19, has more grooves 14 than either the first 31 or second 32 zone to allow the remaining amount laser energy to escape. In a similar manner, the steepness of the pitch in the slit pattern may be varied from shallowest at the proximal zone 31 to the steepest at the distal zone 33. The remaining light energy that has not escaped through any of the zones may be reflected back towards the fiber core 3 due to the concave shape 27 of the outer cap 16, as described above.

The laser generator may generate up to 10 Watts of laser energy, In one embodiment using 5 Watts of power about less than 0.5 Watts of the laser energy will be emitted from the distal tip 19 which results in approximately 4.5 Watts of laser energy that will uniformly and radially be emitted from the radial energy emitting section 4. However, if desired, the amount of laser energy that is released out of the distal tip 19 can be increased by removing the concave distal end 27 from the outer cap 16, changing the angle of the reflective surface 27 or by changing the configuration of the grooves 14.

As shown in FIGS. 5C-5H, various other embodiments of the radial energy emitting section 4 are shown. These various embodiments of the different type of radial energy emitting section 4 are intended to be used with the device embodiment previously described and shown in FIG. 5A. For clarity purposes only FIGS. 5C-5H only depict the fiber core 5 with grooves 14, however it is intended that the other device components described and shown in FIG. 5A would be combined. Referring to FIG. 5C, the grooves 14 are etched into the core 5 in a double helix pattern. A double helix groove 14 configuration consists of two congruent helices with the same axis that differ by translation along the axis. Referring to FIG. 5D, the grooves 14 are etched into the core 5 in a slit or half-moon pattern. In this embodiment the individual grooves 14 may not extend fully around the core 5. Referring to FIG. 5E, the grooves 14 are etched into the core 5 in a dot pattern. Referring to FIG. 5F, the grooves 14 are etched into the core 5 in a longitudinal triangular or wedge pattern.

Referring to FIG. 5G-FIG. 5H, the grooves 14 are etched into the core 5 in a variable pitch pattern. Here, the grooves 14 of the first zone 31 are in a double helix pattern. The grooves 14 of the second zone 32 are also in a double helix pattern but are closer together with a steeper pitch than the grooves 14 of the first zone 31. The grooves 14 of the third zone 33 are also in a double helix pattern and are closer together and more in number than that of the second zone 32. Also, a first space 31a is between the first zone 31 and second zone 32, and a second space 32a is between the second zone 32 and third zone 33. It is understood that the type of groove 14 pattern may differ depending on the desired resulting power density. For example, the first zone 31 may be a double helix, as shown in FIG. 5E, however it is conceived that the second zone 32 groove 14 pattern may be that of slits, as seen in FIG. 5D, and the third zone 33 groove 14 pattern may be a single helix or cork-screw, as seen in FIG. 5A. Referring to FIG. 5H, of the groove 14 pattern for the variable pitch may be circular around the axis of the core 5.

In yet another patter (not shown), it may be possible to have multiple radial energy emitting sections along the length of the device. For such an embodiment sections of the cladding layer may be removed and the exposed core may have grooves etched in any of the patters previously described. The advantage of having multiple radial energy emitting sections along the length of the device is that the treatment time may be reduced because the amount of treatment zones that can have energy delivered will increase.

As seen in FIG. 5I, another embodiment of the device is shown. In this embodiment, the device comprises of a core 5 with a radial energy emitting section 4 having varying pitch grooves 14 as described in FIG. 5G above. This embodiment also has a sleeve 17 coaxially aligned with and secured to the fiber. The sleeve 17 may be made of similar material as described in previous embodiments above, such as glass or fused silica. The distal most end 102 of the sleeve 17 may be a selected distance proximal from the distal most end 100 of the core 5. A sensor 103 may be securely attached to the distal most end 100 of the core 5. An electrical wire 101 may be connected to the sensor 103 and extend back towards the generator (not shown). The purpose of the sensor 103 in this embodiment is to measure the amount of light energy escaping from the front of the core 5 and not escaping from the radial energy emitting section 4. The sensor 103 may measure temperature of the core 5, light wavelengths, light energy, or the temperature of surrounding fluid or tissue. An example of such a sensor 103 is a photodiode sensor used to measure optical power. By measuring the optical power being delivered from the front of the device, and knowing the total wattage being used, it is possible to equate what percentage of the laser energy is being delivered through the radial energy emitting sections 4. An advantage of using a sensor 103 to measure the optical power escaping from the front of the device is that the power wattage may be adjusted to ensure that proper laser energy is being emitted from the radial energy emitting sections 4. The sensor may communicate with a processor within the laser generator which may include an algorithm, or other software component, that can automatically change (either lower or higher) the wattage being delivered to the fiber based on the feedback and information received from the sensor 103. For example, if the sensor 103 is measuring optical power that indicates the light energy delivered by radial energy emitting sections 4 is lower than the power density threshold sufficient for cell death then the system may automatically increase the wattage until the desired power is measured. Therefore, the sensor 103 may act as a feedback mechanism sending information to the generator that can be calculated and the power or wattage may then automatically change (i.e., increased or decreased) depending on the information received. In yet another embodiment, there may be an adjustable second cladding sleeve which can be coaxially advanced or retracted to expose or cover portions of the slits or grooves. This embodiment allows for a single product to be adjusted based on the needs of the clinical users. Advantageously, this allows a manufacturer to produce less inventory and thereby reduce overall product manufacturing costs.

As shown in FIG. 5J, is an image of the light energy being emitted by the device of the embodiment shown in FIG. 5A. The image shows the majority of the light energy being emitted by the radial energy emitting sections 4, as can be seen by the intensity and brightness of this light. The picture also shows that only a small amount of the light energy is being emitted in a forward direction 4a, as can be seen by the low intensity and dullness of this light.

As shown in FIG. 5K, an image of the distal section of the device, as described in previous embodiment FIG. 5A, after it has been used to treat a blood vessel. The fiber 3 and distal portion of sleeve 17 show little to no coagulated blood indicating that any light energy escaping through these portions was not sufficient to thermally induce coagulation and cause cell death. The majority of the clotted blood 105 is shown over the portion of the device that is the radial energy emitting section. This indicates that the power density of the light energy delivered by the radial energy emitting sections 4 was sufficient to thermally induce coagulation and cause cell death. FIG. 5L shows a device currently known in the prior art and is a forward firing laser. The fiber 3 and sleeve 17a of a forward firing device has no blood accumulation because no light energy escapes. However, a large amount of coagulated blood 107 can be seen at the distal most end of the sleeve 17a, indicating the majority of the power density is being delivered in a forward direction.

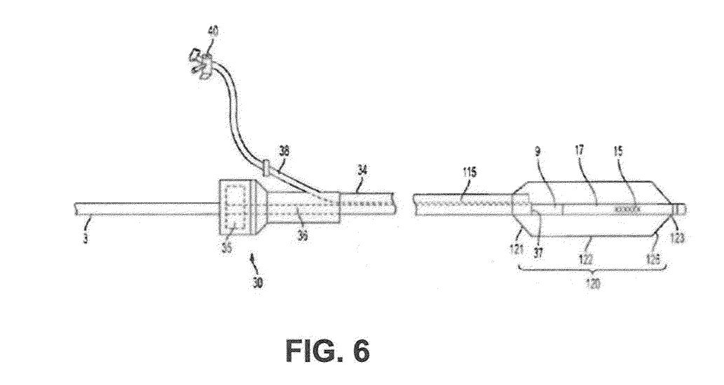

Referring to an alternative embodiment as shown in FIGS. 6-8D, the device 1 may be provided with a spacer 120. Spacer 120 may be expandable, such as an inflatable balloon, expandable basket, expandable arms, cage with expandable arms or non-expandable element, such as an outer ferrule, or a diffuser cap as known in the art.

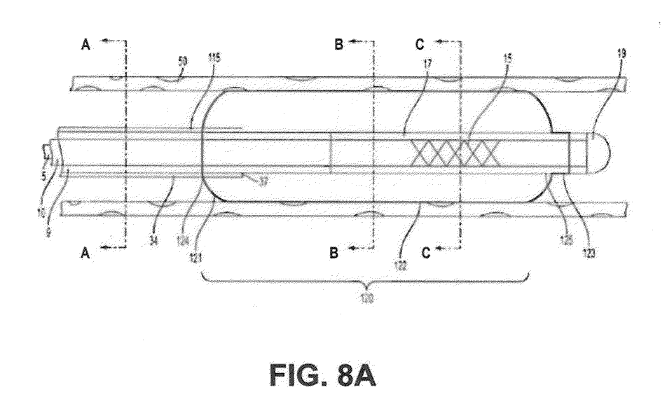

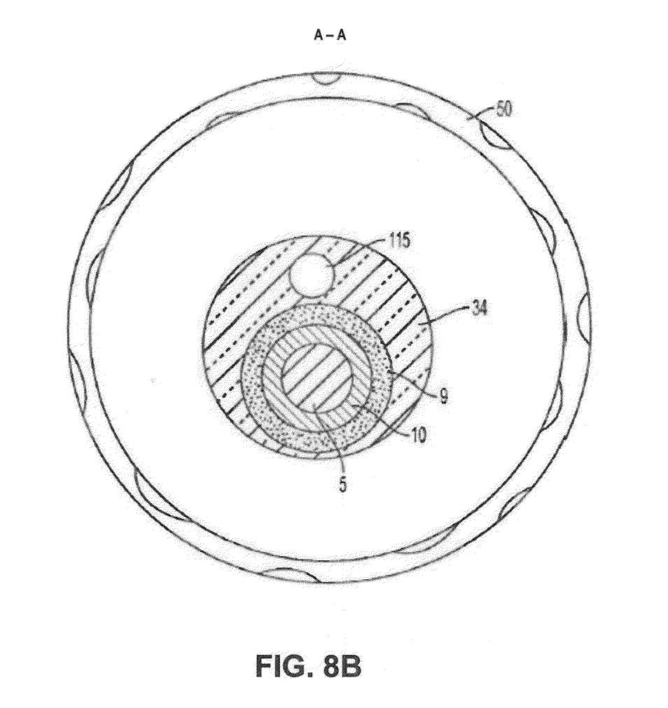

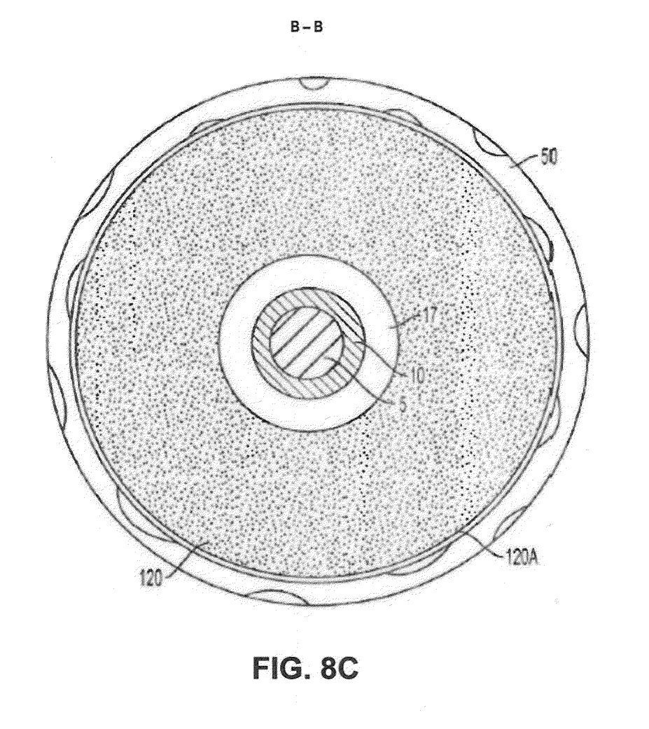

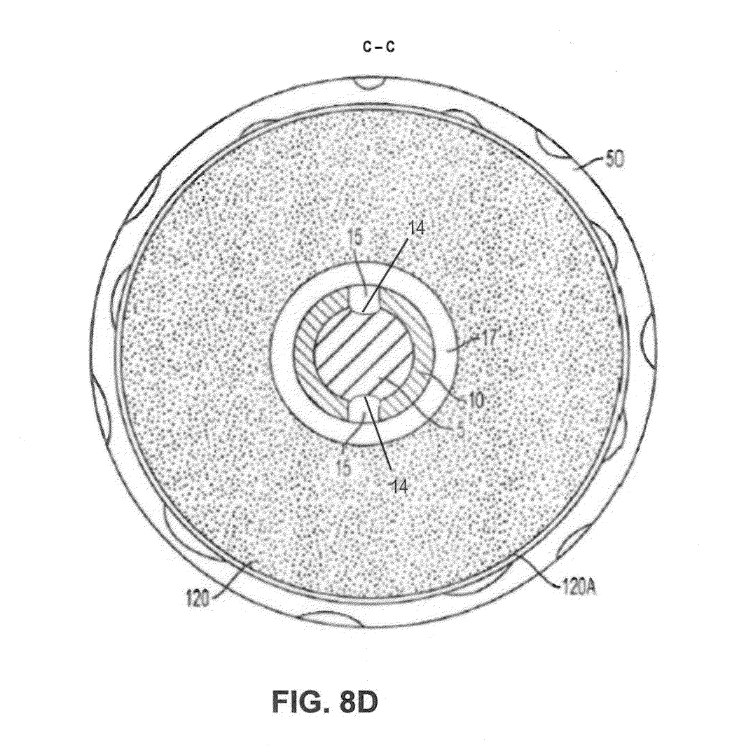

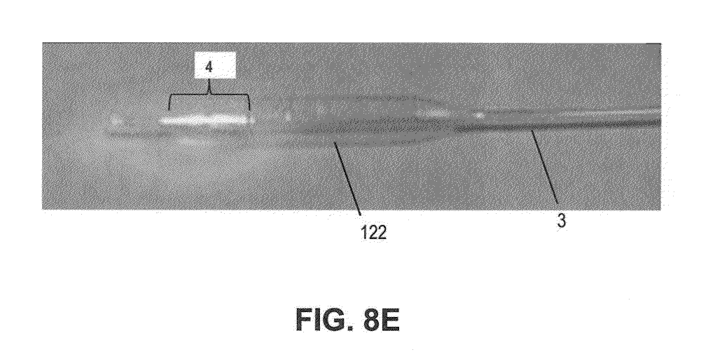

The spacer 120 of this embodiment may be a balloon and may be made out of PTFE, latex or other similar material well-known in the art to make medical grade balloons. The spacer 120 is comprised of a body 122, a distal tapering cone 126, a proximal tapering cone 121, and a distal neck 123. In the deployed state, an outer wall of the spacer 120A (FIG. 8C) at the body 122 of the spacer 120 is in contact with a vessel wall 50. When the spacer 120 is deployed, the slit configuration 15 may be centered within the vein lumen.

FIG. 6 shows the embodiment with a balloon spacer 120 comprising the radial light emitting section 15 of the optical fiber 3 and an outer shaft 34 having a hub 30. The hub 30 may further comprise a homeostasis valve 35, a side arm or Y-connector 38, a stopcock 40, and a through-lumen 36 for insertion and passage of the optical fiber 3 to the outer shaft 34. The outer shaft 34 terminates with the balloon body at the distal tip 37. The side arm 38 is in communications with the inflation/deflation lumen 115 positioned within outer shaft 34 and terminating within the balloon body.

As used herein, the outer shaft 34 can be a sheath, dilator or any other tubular device designed to aid in insertion and advancement of the optical fiber 3 through a blood vessel. The homeostasis valve 35 is a passive one-way valve that prevents the backflow of blood from the through-lumen 36 while simultaneously allowing the introduction of fibers, guidewires, and other interventional device to the outer shaft 34. The valve 35 is located within the lumen 36 of the hub 30. The valve 35 is made of elastomeric material such as a PTFE or silicone, as commonly found in the art. The valve 35 opens to allow insertion of the fiber 3 and then seals around the inserted fiber 3. However, the valve 35 does not open in response to pressure from the distal side of the device in order to prevent back-flow of blood or other fluids. The valve 35 also prevents air from entering the outer shaft 34.

The stopcock 40 and side arm tubing 38 provide multiple fluid/gas paths for administering optional procedural fluids and gases during a treatment session as described in more detail below. The stopcock 40 may be a three-way valve with a small handle (not shown) that can be moved to alter the fluid/gas path. The position of the handle controls the active fluid/gas path by shutting off the flow from one or both ports of the stopcock 40.

The fiber 3 runs coaxially within the through-lumen 36 of the outer shaft 34. During manufacture, the fiber is permanently bonded to the hub 30 using an adhesive or other known technique. Advantageously, the adhesive secures the fiber 3 to the hub 30 so that there can be no independent movement of the fiber 3 relative to the outer shaft 34 during use. When the fiber 3 is inserted through the outer shaft 34 and fiber 3 is bonded to the hub 30, the laser treatment device is in a locked operating position. In that operating position, the fiber tip 19 extends past the distal tip 37 of the outer shaft 34 by a set amount to expose the distal end section 12. The tip 37 ends within the balloon spacer 120 so that it allows carbon dioxide gas to pass through the inflation/deflation lumen 115 from the side-arm lumen 38 where the administration of the carbon dioxide gas is controlled by the stopcock 40.

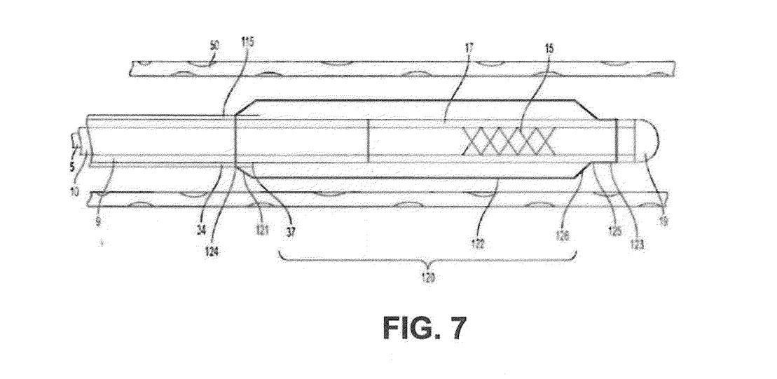

Referring to FIGS. 7-8C, the method of using the above endovascular device embodiment is shown. If the spacer 120 is a balloon, then gas, including but not limited to C02, would likely be used to inflate the balloon because the gas will not lower the energy as light travels through the slits 15 and towards the vein walls. A key aspect of this embodiment is that laser energy is intended to be delivered as close to the inner vessel wall as possible with the lowest amount of power loss possible. Using carbon dioxide gas instead of fluid, such as saline solution, may be advantageous because the laser energy will travel through gas without being absorbed. The laser energy will emit through the sides of the balloon that are in contact with the vessel wall. Carbon dioxide is a safe inflation mass because it is regularly removed by the human body, so if the balloon 120 were to rupture the carbon dioxide could be naturally removed from the body. The expandable spacer 120 is attached or connected onto the outer shaft 34 at proximal bond point 124 and to the sleeve 17 at distal bond point 125.

The outer shaft 34 may be a dual lumen catheter having an inflation/deflation lumen 115 and a second lumen sufficient for passage of the fiber 3 as shown in FIG. 8B, which depicts a cross-sectional view along line A-A′ in FIG. 8A. The fiber 3 is shown positioned inside vessel 50. The device is comprised of an outer shaft 34 including an inflation lumen 115 positioned within the wall of the shaft 34. Within the fiber lumen is shown the components of the fiber; the jacket 9, cladding 10 and core 5. FIG. 8C represents a cross-sectional view along line B-B′ where the core 5 is coaxially surrounded by a portion of the cladding 10 having no slits and core 5 having no grooves. At this point, the cladding 10 is surrounded by the glass sleeve 17 instead of the protective jacket 9, which has been removed from this section of the fiber. The glass sleeve is coaxially surrounded by the inflated balloon 120 which touches the wall of the vein lumen 50.

FIG. 8D represents a cross-sectional view along the line C-C′ at the midpoint of the balloon body 122. Here, the laser energy escapes from the core 5 through the grooves 14 and the slits 15 in the cladding. The laser energy travels through the glass sleeve 17 and the CO2 in the balloon 120 with little to no loss in power density because neither sleeve 17 nor CO2 will absorb the light wavelength. The laser energy will be absorbed by the vessel wall 50 which is in contact with the outer wall of the balloon 120A. An advantage of this embodiment is that a large percentage of power density is being directly absorbed by the vessel wall 50 because neither the sleeve 17 nor CO2 absorb the light wavelength. This means that the device does not need to deliver as high a power density as forward firing lasers or radial lasers in the art which rely on radiant heating (i.e., heating the blood first and this heat energy is the transferred to the vein wall).

As shown in FIG. 8E, an image of the embodiment described above and shown in FIGS. 6-8D. The image shows the radial energy emitting section 4 emitting laser energy while the balloon spacer 122 is inflated.

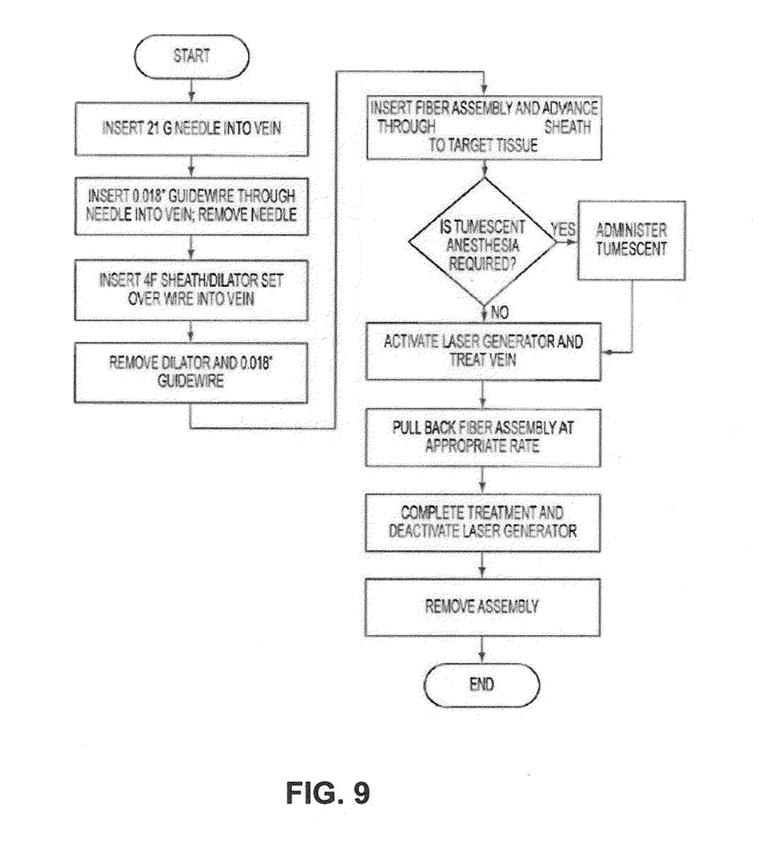

Methods of using the optical fiber device for endovenous treatment of varicose veins and other vascular disorders will now be described with reference to FIG. 9, which illustrates the procedural steps associated with performing endovenous treatment using the optical fiber device 1. To begin the procedure, the target vein is accessed using a standard Seldinger technique well known in the art. Under ultrasonic guidance, a small gauge needle is used to puncture the skin and access the vein. A 0.018 inch guidewire is advanced into the vein through the lumen of the needle. The needle is then removed leaving the guidewire in place.

A micropuncture sheath/dilator assembly is then introduced into the vein over the guidewire. A micropuncture sheath dilator set, also referred to as an introducer set, is a commonly used medical kit, for accessing a vessel through a percutaneous puncture. The micropuncture sheath set includes a short sheath with internal dilator, typically 5-10 cm in length. This length is sufficient to provide a pathway through the skin and overlying tissue into the vessel, but not long enough to reach distal treatment sites. Once the vein has been accessed using the micropuncture sheath/dilator set, the dilator and 0.018 inch guidewire are removed, leaving only the micropuncture introducer sheath in place within the vein. A 0.035 inch guidewire is then introduced through the introducer sheath into the vein. The guidewire is advanced through the vein until its tip is positioned near the sapheno-femoral junction or other starting location within the vein.

After removing the micropuncture sheath, a treatment sheath/dilator set is advanced over the 0.035 inch guidewire until its tip is positioned near the sapheno-femoral junction or other reflux point. Unlike the micropuncture introducer sheath, the treatment sheath is of sufficient length to reach the location within the vessel where the laser treatment will begin, typically the sapheno-femoral junction. Typical treatment sheath lengths are 45 and 65 cm. Once the treatment sheath/dilator set is correctly positioned within the vessel, the dilator component and guidewire are removed from the treatment sheath.

The optical fiber assembly 1 is then inserted into the treatment sheath lumen and advanced until the fiber assembly distal end is flush with the distal tip of the treatment sheath. A treatment sheath/dilator set as described in U.S. Pat. No. 7,458,967, incorporated herein by reference, may be used to correctly position the protected fiber tip with spacer assembly 1 of the current invention within the vessel. The treatment sheath is retracted a set distance to expose the fiber tip, typically 1 to 2 cm. If the fiber assembly has a connector lock as described in U.S. Pat. No. 7,033,347, also incorporated herein by reference, the treatment sheath and fiber assembly are locked together to maintain the 1 to 2 cm fiber distal end exposure during pullback, as seen in FIG. 6.

At this time, prior art methods require the administration of tumescent anesthesia along the vein, which can take up to 30 minutes. The present invention emits laser energy radially, directing the energy to the vessel wall and as a result, only requires a low power density, which eliminates perforations and thermal damage to surrounding tissue and nerves. Therefore the present invention may not require the administration of tumescent anesthesia. However, if tumescent is required then the physician may inject at this time.

Once device 1 has in proper treatment position relative to the sapheno-femoral junction, the laser generator 2 is turned on and the laser light enters the optical fiber 3 from its proximal end via the proximal connection to the laser generator 7. While the laser light is emitting laser light through the distal end section 4, the treatment sheath/fiber assembly is withdrawn through the vessel at a variable rate, ranging at 50-80 J/cm for 2-3 millimeters per second, and also depending on the size of the vessel being treated. Alternatively, in another embodiment of the method the physician may withdraw the sheath/fiber assembly in a pulsed manner. The laser energy travels along the optical fiber 3 through the slits 15 and into the vein lumen where the laser energy is uniformly delivered radially to heat the vein wall, thus damaging the vein wall tissue, causing cell necrosis and ultimately causing collapse/occlusion of the vessel. Forward firing of the lasers which require high power densities to boil or heat the blood, creating bubbles which are necessary for 360 degree circumferential treatment of the targeted vein. High power densities can cause perforations, bruising, nerve damage, thermal damage to non-targeted tissue and other complications causing the patient additional pain. High power densities also cause charring of blood on the fiber tip. Advantageously, the method of using this invention does not require high power density in a forward firing direction and therefore these risks are diminished or removed from the treatment.

The outer jacket 9 of fiber 3 may include visual markings/markers. Markings are used by the physician to provide a visual indication of insertion depth, tip position and speed at which the device is withdrawn through the vessel during delivery of laser energy. The markings may be numbered to provide the physician with an indication as to distance from the distal end section of the fiber 12 to the access site during pullback. The markings may be positioned around the entire circumference of the fiber shaft or may cover only a portion of the shaft circumference.

Once the targeted tissue is treated, the laser generator 2 is turned off. The procedure for treating the varicose vein is considered to be complete when the desired length of the great saphenous vein has been exposed to laser energy. Normally, the laser generator is turned off when the fiber tip 19 is approximately 3 centimeters from the access site. The combined sheath/endovascular laser treatment device 1 is then removed from the body as a single unit.

Prior art methods provide a cladding that does not have slits therethrough and thus delivers laser energy via an emitting face at the distal tip of the fiber which causes charring and blood build-up on the tip. By emitting laser energy through the slits 15, the device provides radial treatment and reduces the laser energy emitted out of the distal tip 19. Because minimal energy is emitted from the distal tip 19, treatment using the present invention does not result in charring.

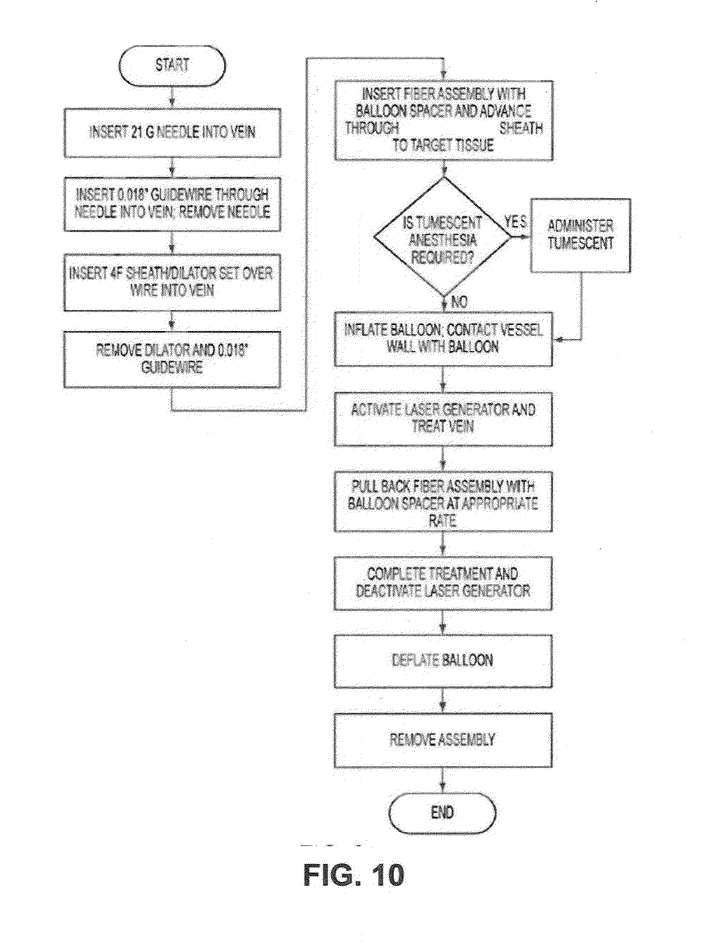

Methods of using the optical fiber device with balloon spacer for endovenous treatment of varicose veins and other vascular disorders will now be described with reference to FIG. 10, which illustrates the procedural steps associated with performing endovenous treatment using this embodiment of the optical fiber device 1. Using much of the same steps as the previous method, the optical fiber 3 is inserted and advanced to the treatment location with a balloon 120 in the deflated position as shown in FIG. 8A. If tumescent anesthesia is required, the physician should administer it after the fiber has been advanced to the treatment location. However, the hub 30 and catheter 34 enable the filling of the balloon 120 via the stopcock 40 and side-arm 38 which defines the inflation deflation lumen 115. Prior to activating the laser generator, the balloon 120 is deployed by injecting inflation gas through the inflation lumen 115 into the balloon 120 as shown in FIGS. 7-8A. As the gas fills the balloon 120 it expands and the outer wall of the expandable member 120 contacts the inner vessel wall 50 centering the radial energy emitting section 4 within the vein lumen. The deployed balloon 120 maintains the position of the distal end section 12 of the fiber 3 within the vein lumen and out of contact with the vessel wall.

In this embodiment, markings can be placed on the catheter 34 instead of jacket 9, as in the previous embodiment so that the physician can measure the rate at which the fiber 3 is being pulled back. The catheter 34/fiber 3 assembly is slowly withdrawn together through the vein. The connection between the fiber connector 31 and hub connector 32 ensures that the distal end section 4 remains exposed beyond the catheter tip 37 by the recommended length for the entire duration of the treatment procedure. Once treatment is complete, the expandable member 120 is deflated and device is removed. This embodiment has the ability to inflate and/or deflate as the device is moved through the vessel to accommodate varying diameter vein segments.

As may be recognized by those of ordinary skill in the pertinent art, blood vessels other than the great saphenous vein and other hollow anatomical structures can be treated using the device and/or methods of the invention disclosed herein.

The above disclosure is intended to be illustrative and not exhaustive. This description will suggest many modifications, variations, and alternatives that may be made by those of ordinary skill in this art without departing from the scope of the invention. Those familiar with the art may recognize other equivalents to the specific embodiments described herein. Accordingly, the scope of the invention is not limited to the foregoing specification.

1. An endovascular laser treatment device for causing closure of a blood vessel comprising:
an optical fiber adapted to be inserted into a blood vessel and having a core through which a laser light travels;
a cladding layer coaxially surrounding the optical fiber core;
a distal portion comprising slits in the cladding; and
wherein a cap is arranged around the distal portion.

2. The endovascular laser treatment device of claim 1 further comprising grooves in the core.

3. The endovascular treatment device of claim 2, wherein the slits align with the grooves.

4. The endovascular treatment device of claim 1, wherein the cap is a glass ferrule.

5. The endovascular treatment device of claim 1, wherein the cap comprises a concave shape near its distal end.

6. The endovascular treatment device of claim 1 further comprising an air gap between the cap and the core.

7. The endovascular treatment device of claim 3 further comprising an ablation section.

8. The endovascular treatment device of claim 7, wherein the ablation section is further comprised of the grooves.

9. The endovascular treatment device of claim 7, wherein the grooves further comprise of a first zone, a second zone, and a third zone.

10. The endovascular treatment device of claim 9, wherein the first zone has fewer grooves than the second zone.

11. The endovascular treatment device of claim 10, wherein the second zone has fewer grooves than the third zone.

12. An endovascular treatment device for treating a varicose vein comprising of:
an optical fiber adapted to be inserted into a blood vessel and having a core through which a laser light travels; a cladding layer coaxially surrounding the optical fiber core; a distal portion of the optical fiber core having grooves; and a spacer element.

13. The endovascular treatment device of claim 12, wherein the spacer element is an inflatable balloon having an outer wall.

14. The endovascular treatment device of claim 12 further comprising grooves in the core.

15. The endovascular treatment device of claim 14, wherein the grooves align with the slits.

16. The endovascular treatment device of claim 13, wherein the balloon is inflated with a gas.

17. An endovascular treatment device for treating a varicose vein comprising of:
an optical fiber adapted to be inserted into a blood vessel and having a core through which a laser light travels; a cladding layer coaxially surrounding the optical fiber core; a distal portion of the optical fiber core having grooves; and a cap coaxially surrounding the distal portion of the core.

18. The endovascular treatment device of claim 17, wherein the cap is a glass ferrule.

19. The endovascular treatment device of claim 17 further comprising a sensor and a power source.

20. The endovascular treatment device of claim 19, wherein the sensor measures the light energy escaping from the core and the power source automatically adjusts the amount of laser energy being delivered to the fiber based on the sensor measurements.
